(19) United States
(12) Patent Application Publication
(10) Pub. No.: US 2024/0398651 A1
(43) Pub. Date: Dec. 5, 2024

(54) ISCHEMIC STROKE REHABILITATION PROTOCOL IMPLEMENTING ROBOTIC ASSISTED REHABILITATION IN SUBJECTS WITH PHARMACOLOGICALLY INDUCED NEUROPLASTICITY AND OTHER CONDITION-BASED TARGETED SYNAPTIC REBUILDING OR ENHANCEMENT PROTOCOLS IMPLEMENTING ROBOTIC ELECTRICAL AND ELECTROMAGNETIC STIMULI IN SUBJECTS WITH PHARMACOLOGICALLY INDUCED NEUROPLASTICITY

(71) Applicant: Neuro-Innovators, LLC., Sewickley, PA (US)

(72) Inventors: Peter C. Doyle, Sherborn, MA (US); John Howison Schroeder, Sewickley, PA (US)

(21) Appl. No.: 18/677,635

(22) Filed: May 29, 2024

Related U.S. Application Data
(63) Continuation of application No. PCT/US2022/051298, filed on Nov. 29, 2022.
(60) Provisional application No. 63/283,755, filed on Nov. 29, 2021.

(51) Int. Cl. A61H 1/02; A61K 31/155; A61K 31/381; A61K 31/4184; A61K 31/4709

(52) U.S. Cl. CPC A61H 1/0288; A61K 31/155; A61K 31/381; A61K 31/4184; A61K 31/4709; A61H 2201/105

(57) ABSTRACT

A stroke rehabilitation protocol comprising the steps of: providing a subject with medicaments including at least one of Cilostazol, Metformin, Telmisartan and Duloxetine; and implementing robotic assisted rehabilitation in the subject with pharmacologically induced neuroplasticity, wherein the medicaments are supplied in effective amounts and timing whereby the medicaments induce neuroplasticity during the robotic assisted rehabilitation. The stroke rehabilitation protocol includes multiple robotic assisted rehabilitation sessions and the medicaments are orally administered to the patient within 4 hours implementing of each robotic assisted rehabilitation session. The multiple robotic assisted rehabilitation sessions may be on an upper limb robot with 500-700 repetitions per session which sessions total at least 10 hours.

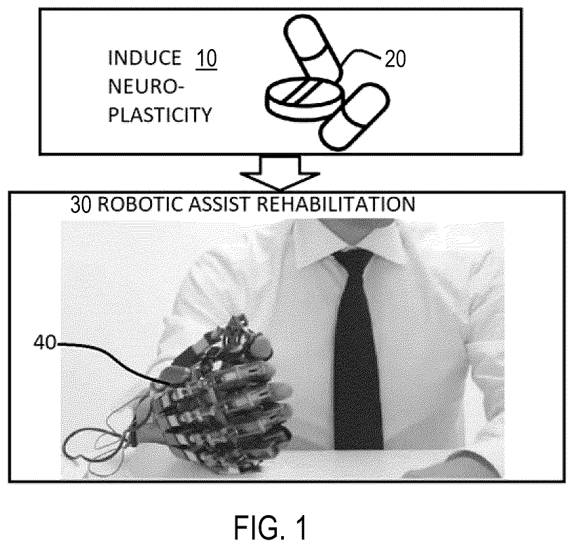

### RELATED APPLICATIONS

The present application is a continuation of international patent application PCT/US2022/051298 that published Jun. 1, 2023 as publication WO 2023/097123, which is incorporated herein by reference. International patent application PCT/US2022/051298 claims the benefit of U.S. provisional patent application Ser. No. 63/283,755 filed Nov. 29, 2021 titled “Targeted Synaptic Rebuilding or Enhancement Implementing Robotic Electrical and Electromagnetic Stimuli in Subjects With Pharmacologically Induced Neuroplasticity”

### BACKGROUND OF THE INVENTION

### 1. Field of the Invention

This invention generally relates to neurological engagement methods for targeted synaptic rebuilding or enhancement utilizing but not limited to, robotic, electrical and or electromagnetic stimuli in subjects with pharmacologically induced neuroplasticity and systems for implementing the same. Specifically, the present invention is directed to ischemic stroke rehabilitation protocol implementing robotic assisted rehabilitation in subjects with pharmacologically induced neuroplasticity.

### 2. Background Information

Neurological rehabilitation regimes implementing neuroplasticity have recently begun to be successfully implemented. See Sidyakina I. V. et al The Mechanism of Neuroplasticity and rehabilitation in stroke acuity, Annals of Neurology, Vol. 7 No. 1, 2013 pp 52-56. See also Kraus Christoph et al, Serotonin and Neuroplasticity—Links between molecular, functional and structural pathophysiology in Depression, Neuroscience and Bio-behavioral Review, 2017, volume 77, pp 317-326. See also López-Valdés HE, Clarkson AN. Ao Y. Charles AC, Carmichael ST, Sofroniew MV, Brennan KC. Memantine enhances recoveryfrom stroke. Stroke. 2014 July; 45(7):2093-2100.

Neurological rehabilitation of subjects with pharmacologically induced neuroplasticity using oculomotor visual tasks with a video-occulography (VOG) system is disclosed in WO 2020/097320, which application is incorporated herein by reference (see also U.S. Patent Publication 2021-0251555).

Robotic assisted rehabilitation, also called robotic-mediated therapy, is a form of rehabilitation that enables highly repetitive, intensive, adaptive, and quantifiable physical training. Robotic devices used for motor rehabilitation include end-effector and exoskeleton types. It has been used to restore loss of motor function, mainly in stroke survivors suffering from an upper limb paresis. Multiple studies collated in a growing number of review articles showed the positive effects on motor impairment, less clearly on functional limitations. See early work in this area from over twenty years ago: Aisen ML, Krebs HI, Hogan N, McDowell F. Volpe BT. The effect of robot-assisted therapy and rehabilitative training on motor recovery following stroke. Arch Neurol. (1997) 54:443-6; and Volpe BT, Krebs HI, Hogan N, Edelstein L. Diels C. Aisen ML. A novel approach to stroke rehabilitation: robot-aided sensorimotor stimulation. Neurology. (2000) 54:1938-44. Additionally See also Masiero S. Armani M. Rosati G. Upper-limb robot-assisted therapy in rehabilitation of acute stroke patients: focused review and results of new randomized controlled trial. J Rehabil Res Dev. (2011) 48:355-66; Sale P, Franceschini M, Mazzoleni S, Palma E, Agosti M. Posteraro F, et al. Effects of upper limb robot-assisted therapy on motor recovery in subacute stroke patients. J Neuroeng Rehabil. (2014) 11:104; Wu X, Guarino P, Lo AC, Peduzzi P. Wininger M. Long-term effectiveness of intensive therapy in chronic stroke. Neurorehabil Neural Repair. (2016) 30:583-90; and Bertani R. Melegari C, De Cola MC, Bramanti A. Bramanti P, Calabrb RS. Effects of robot-assisted upper limb rehabilitation in stroke patients: a systematic review with meta-analysis. Neurol Sci. (2017) 38:1561-9.

Additionally, there is a growing use of other electrical and electromagnetic stimuli within the nervous system to address neuro-dysfunction resulting from limb loss, neurological trauma or neurodegenerative diseases. Consider, for example, repetitive Transcranial Magnetic stimulation which has been shown to reduce the negative symptoms of schizophrenia and depression, which is discussed in U.S. Publication 2011-0112427, which application is incorporated herein by reference.

Repetitive transcranial magnetic stimulation (rTMS) is a form of brain stimulation therapy used to treat depression and anxiety. It has been in use since 1985. The therapy involves using a magnet to target and stimulate certain areas of the brain.

Replacing the function of a missing or paralyzed limb with a prosthetic device that acts and feels like one's own limb/restores motion to paralyzed limb is a major goal in applied neuroscience. Studies in nonhuman primates have shown that motor control and sensory feedback can be achieved by connecting sensors in a robotic arm to electrodes implanted in the brain. See Kelly Collins et al Ownership of an artificial limb induced by electrical brain stimulation, Proceedings of the National Academy of Sciences of the United States of America Jan. 3, 2017, 114(1) 166-171. See also WO 2001-002054 relates to a system and method for relieving phantom limb pain in amputees, and for providing an amputee with sensory feedback from a prosthetic limb which application is incorporated herein by reference.

Cranial electrotherapy stimulation (CES) is a form of neurostimulation that delivers a small, pulsed, alternating current via electrodes on the head. CES is used with the intention of treating a variety of conditions such as anxiety, depression and insomnia. CES has been suggested as a possible treatment for headaches, fibromyalgia, smoking cessation, and opiate withdrawal.

Deep brain stimulation (DBS) is a neurosurgical procedure involving the placement of a medical device called a neurostimulator (sometimes referred to as a “brain pacemaker”), which sends electrical impulses, through implanted electrodes, to specific targets in the brain (brain nuclei) for the treatment of movement disorders, including Parkinson's disease, essential tremor, and dystonia and other conditions such as obsessive-compulsive disorder and epilepsy. While its underlying principles and mechanisms are not fully understood, DBS directly changes brain activity in a controlled manner. DBS has been approved by the Food and Drug Administration as a treatment for essential tremor and Parkinson's disease (PD) since 1997. DBS was approved for dystonia in 2003, obsessive-compulsive disorder (OCD) in 2009, and epilepsy in 2018. DBS has been studied in clinical trials as a potential treatment for chronic pain for various affective disorders, including major depression.

Transcranial direct current stimulation (tDCS) is a form of neuromodulation that uses constant, low direct current delivered via electrodes on the head. It was originally developed to help patients with brain injuries or neuropsychiatric conditions such as major depressive disorder. It can be contrasted with cranial electrotherapy stimulation, which generally uses alternating current the same way, as well as transcranial magnetic stimulation. Research shows increasing evidence for tDCS as a treatment for depression. There is mixed evidence about whether tDCS is useful for cognitive enhancement in healthy people. There is evidence that tDCS, alone in its current implementation, is not useful for memory deficits in Parkinson's disease and Alzheimer's disease, non-neuropathic pain, nor for improving arm or leg functioning and muscle strength in people recovering from a stroke.

Functional electrical stimulation (FES) is a technique that uses low-energy electrical pulses to artificially generate body movements in individuals who have been paralyzed due to injury to the central nervous system. More specifically, FES can be used to generate muscle contraction in otherwise paralyzed limbs to produce functions such as grasping, walking, bladder voiding and standing. This technology was originally used to develop neuroprostheses that were implemented to permanently substitute impaired functions in individuals with spinal cord injury (SCI), head injury, stroke and other neurological disorders. In other words, a person would use the device each time he or she wanted to generate a desired function. FES is sometimes also referred to as neuromuscular electrical stimulation (NMES). FES technology has been used to deliver therapies to retrain voluntary motor functions such as grasping, reaching and walking. In this embodiment, FES is used as a short-term therapy, the objective of which is restoration of voluntary function and not lifelong dependence on the FES device, hence the name functional electrical stimulation therapy, FES therapy (FET or FEST). In other words, the FEST is used as a short-term intervention to help the central nervous system of the person to re-learn how to execute impaired functions, instead of making the person dependent on neuroprostheses for the rest of her or his life.

Vagus nerve stimulation (VNS) is a medical treatment that involves delivering electrical impulses to the vagus nerve. It is used as an add-on treatment for certain types of intractable epilepsy and treatment-resistant depression.

Responsive neurostimulation (RNS) is a surgical approach to treating seizures that are not controlled by medication. A neurostimulator is placed under the scalp and within the skull, and it is connected to 2 electrodes placed either on the surface of the brain, into the brain, or a combination of both. The device continuously monitors brain activity and then is programmed to detect seizures. When a seizure or seizure-like activity is detected, the device delivers a small amount of electrical current to the brain to stop or shorten the seizure, or possibly prevent a seizure altogether.

From the above, it is evident that there is a need for effective neurological rehabilitation methods and training methods for targeted synaptic rebuilding or enhancement utilizing robotic, electrical and electromagnetic stimuli in subjects.

### SUMMARY OF THE INVENTION

One embodiment of the present invention provides a stroke rehabilitation protocol comprising the steps of: providing a subject with medicaments including at least one of Cilostazol, Metformin, Telmisartan and Duloxetine; and implementing robotic assisted rehabilitation in the subject with pharmacologically induced neuroplasticity, wherein the medicaments are supplied in effective amounts and timing whereby the medicaments induce neuroplasticity during the robotic assisted rehabilitation.

The stroke rehabilitation protocol according to one embodiment of the present invention provides wherein implementing robotic assisted rehabilitation includes multiple robotic assisted rehabilitation sessions. The implementing robotic assisted rehabilitation may include multiple robotic assisted rehabilitation sessions on an upper limb robot with 500-700 repetitions per session, and wherein implementing robotic assisted rehabilitation includes the multiple robotic assisted rehabilitation sessions which total at least 10 hours.

The stroke rehabilitation protocol according to one embodiment of the present invention provides wherein the medicament is orally administered to the patient within 4 hours and preferably within 2 hours of implementing of each robotic assisted rehabilitation session.

The stroke rehabilitation protocol according to one embodiment of the present invention provides wherein implementing robotic assisted rehabilitation includes multiple robotic assisted rehabilitation sessions and Cilostazol is orally administered to the patient in amounts of less than 50 Mg per robotic assisted rehabilitation session.

The stroke rehabilitation protocol according to one embodiment of the present invention provides wherein implementing robotic assisted rehabilitation includes multiple robotic assisted rehabilitation sessions and Metformin is orally administered to the patient in amounts of less than 250 Mg per robotic assisted rehabilitation session.

The stroke rehabilitation protocol according to one embodiment of the present invention provides wherein the medicament includes Cilostazol and one of Telmisartan and Duloxetine, and wherein implementing robotic assisted rehabilitation includes multiple robotic assisted rehabilitation sessions and Cilostazol and one of Telmisartan and Duloxetine are orally administered to the patient within 4 hours implementing of each robotic assisted rehabilitation session.

The stroke rehabilitation protocol according to one embodiment of the present invention provides wherein the medicament includes Metformin and one of Telmisartan and Duloxetine, and wherein implementing robotic assisted rehabilitation includes multiple robotic assisted rehabilitation sessions and Metformin and one of Telmisartan and Duloxetine are orally administered to the patient within 4 hours implementing of each robotic assisted rehabilitation session.

### BRIEF DESCRIPTION OF THE FIGURE

The FIGURE schematically illustrates an ischemic stroke rehabilitation protocol implementing robotic assisted rehabilitation in subjects with pharmacologically induced neuroplasticity in accordance with one embodiment of the present invention.

### DESCRIPTION OF THE PREFERRED EMBODIMENTS

The various embodiments and examples of the present invention as presented herein are understood to be illustrative of the present invention and not restrictive thereof and are non-limiting with respect to the scope of the invention.

The overall concept of the present invention provides a method for enhancing success in the restoration of neuro-function in the neurological domain, hereafter referred to as Targeted Synaptic Rebuilding and Neuro-Rehabilitation Enhancement (TSR-NRE). The fundamental principle of the method is the combination of any neurologic task used during therapeutic rehabilitation of neuro-function, with pharmacology which induces neuroplasticity and physiological robotic assisted, electrical or electromagnetic stimuli that promote neuro-rehabilitation success or enhance the rehabilitation effect. Stimulation of neuroplasticity is at the heart of this combinatorial therapy with the goal of establishing and modulating new synaptic pathways to regain neural function lost through illness or injury or gain new neural function for the purpose of enhanced learning, cognition or human performance.

The TSR-NRE method in accordance with the present invention may be described as following general principles of operation: In the presence of a neurological dysfunction, disorder or desired neurological outcome and the presence of an implanted or external electrical or electromagnetic stimulator including robotic assist devices) apply a therapeutic intervention to increase neuroplasticity; During application of the therapy, perform simultaneous tasks as rehabilitation for the dysfunction or disorder and or enhancement of the implanted stimuli. These tasks are precisely controlled by a combined software and hardware system that delivers stimuli according to a protocol of the clinician's discretion; Periodically (on the scale of hours, days, weeks, or according to clinical schedule) assess the success of therapeutic intervention by applying a specific set of tests that objectively assess the neuro-motor, sensory, cognitive or other neurological performance targeted. Assessment may also be performed immediately or in real-time during task performance; and During the task, immediate visual feedback may be provided to the patient to further enhance learning and provide real-time immediate assessment. This feedback may be customized with feedback target goals.

### Neuroplasticity

Neuroplasticity describes the composite changes in wiring in human brains in response to stimulations, exercises, and experiences, such as learning to play an instrument or recovering the use of one's arm with therapy after a stroke. There is a long history in the published literature of doctors observing such changes, but its recognition was largely anecdotal, based on individual observations. Recent advances have enabled detection and measurement of neuronal changes and growth in animal brains, providing us with the ability to observe and understand neuroplasticity in humans. There are numerous published studies showing tangible positive changes in post-stroke patients (referred to as chronic stroke patients) and others with degenerative brain challenges, in response to various treatment regimes.

Neuroplasticity effectively creates a pliable substrate within which rehabilitation can act, allowing individual neurons, synapses, and whole neural networks to experience enhanced and efficient reconfiguration. Rehabilitation should ideally take advantage of this plasticity by exercising, stimulating, and enhancing configurations that are beneficial and therapeutic to the patient. Because of this, targeted stimuli and rehabilitative tasks that exercise valuable functions as well as broad general regions of neural territory are a logical choice.

The term “neuroplasticity” in the context of this application is meant to cover all interpretations of plasticity, or modifiability, in the brain, such as: Neurogenesis, or the creation of new neurons; Apoptosis, or the selective elimination of neurons, which is a normal part of neural re-wiring; Synaptogenesis, or the creation of new, or enhancement of existing (but not yet signaling) synapses between neurons, including branching or pruning of neural or axonal arbors; Synaptic plasticity, namely changes in the communication strength of synapses, either increasing in strength, decreasing, becoming more or less inhibited, or any other beneficial change or modulation of synapses; Changes induced by interactions with, or other changes in, other non-neural cells in the brain, e.g., glia or the peri-neural network (PNN); Changes in genetic expression, e.g., changes in expression of genes that affect brain activity, including but not limited to changes in cell receptors, neurotransmitters, or cell-signaling pathways; and or Changes at the interface of native human tissue and implanted medical devices.

### Ischemic Stroke Rehabilitation Protocol

One preferred embodiment of the present invention is directed to ischemic stroke rehabilitation protocol implementing robotic assisted rehabilitation in subjects with pharmacologically induced neuroplasticity. The ischemic stroke rehabilitation protocol according to the present invention is broadly schematically illustrated in the FIGURE and comprises the steps of: providing at step 10 a patient or subject with medicaments 20, with the medicaments 20 including at least one of Cilostazol, Metformin, Telmisartan and Duloxetine; and implementing a robotic assisted rehabilitation 30 in the subject with a robot 40 and, wherein the medicaments 20 are supplied in effective amounts and timing whereby the medicaments 20 induce neuroplasticity during the robotic assisted rehabilitation 40. The step 10 of providing a subject with medicaments 20 is synonymous with administering the medicaments 20 to the patient and the patient taking of the medicaments 20. As discussed below the preferred delivery method is orally but others are possible.

Regarding chronic stroke patients, these patients are sometimes considered as medically neglected despite being a population of approximately 7 million Americans, and this is a cohort that is unfortunately expanding. Recent statistics from the CDC report that there are 795,000 strokes suffered in the US annually, of which 76% of the patients survive. Yet surviving is not thriving, given that most patients will be disabled due to neural damages in their brains. This reduces the life expectancy of chronic stroke patients by approximately ten years. This creates hardships on families, communities, and the nation, in terms of lost wages, lower productivity, and high medical costs, as well as significantly reducing the quality of life for all involved. The ischemic stroke rehabilitation protocol implementing robotic assisted rehabilitation in subjects with pharmacologically induced neuroplasticity is intended to objectively reduce lost wages and lowered productivity of patients, and reduce the associated high medical costs, as well as significantly improving the quality of life for all involved (when the patients quality of life improves so does the family and support members around the patient)

### Cilostazol for Stroke Protocol

Cilostazol has a formula C₂₀H₂₇N₅O₂ and is a selective inhibitor of phosphodiesterase, which in turn increases the activation of intracellular cAMP and thereby inhibits platelet aggregation. An increase in cAMP results in an increase in the active form of protein kinase A (PKA), which is directly related with an inhibition in platelet aggregation. PKA also prevents the activation of an enzyme (myosin light-chain kinase) that is important in the contraction of smooth muscle cells, thereby exerting its vasodilatory effect.

Cilostazol has been noted as a powerful alternative to aspirin in certain aspects. In previous clinical trials for example, cilostazol has been found to significantly reduce the incidence of recurrent stroke, with fewer hemorrhagic events, compared with aspirin. See Huang Y, Cheng Y, Wu J, Li Y, Xu E, Hong Z, et al; Cilostazol versus Aspirin for Secondary Ischaemic Stroke Prevention Cooperation Investigators. Cilostazol as an alternative to aspirin after ischaemic stroke: a randomised, double-blind, pilot study. Lancet Neurol. 2008; 7:494-499. See also Nakamura T, Tsuruta S, Uchiyama S. Cilostazol combined with aspirin prevents early neurological deterioration in patients with acute ischemic stroke: a pilot study. J Neurol Sci. 2012; 313:22-26.

As noted above one embodiment of the present invention includes providing at step 10 a patient or subject with medicaments 20, with the medicaments 20 including at least one of Cilostazol, Metformin, Telmisartan and Duloxetine, wherein the medicaments 20 are supplied in effective amounts and timing whereby the medicaments 20 induce neuroplasticity during the robotic assisted rehabilitation 40. Cilostazol has an elimination half-life of 11-13 hours and the effective timing of Cilostazol as a medicament 20 (alone or in combination with the other medicaments 20) is within 6 hours of the implementing of a robotic assisted rehabilitation 30 session by the subject with the robot 40. More preferably Cilostazol is orally administered to the patient within 4 hours and most preferably within 2 hours (often about one hour) of the implementing of a robotic assisted rehabilitation 30 session by the subject with the robot 40.

The Cilostazol may have several delivery methods or routes of administration, but oral is preferred. Effective amounts of Cilostazol in the protocol of the present invention is less than 200 Mg, more preferably less than 100 Mg, and most preferably less than 50 Mg per robotic assisted rehabilitation 30 session by the subject with the robot 40. Where the medicament 20 includes Cilostazol with one or more of the other cited drugs the effective amounts of Cilostazol in the protocol of the present invention is about 25 Mg (wherein about within this application is +/−10%), possibly less than 25 Mg per robotic assisted rehabilitation 30 session by the subject with the robot 40.

Cilostazol is often administered to patients to treat the symptoms of intermittent claudication, and their dosage typically is 200 Mg per day in a twice per day oral treatments. Where a patient of the present protocol is taking 200 Mg of Cilostazol per day for other reasons, like treatment of blood flow disorders, then this prescribed about is the effective amount for the protocol of the present invention and no additional Cilostazol is required, but this Cilostazol should still be taken within the effective time of the robotic assisted rehabilitation 30 session by the subject with the robot 40.

The protocol of the present invention is believed to yield synergistic effects with combinations of the drugs to form the medicament 20. The preferred combination of the medicaments 20 includes Cilostazol, Metformin, and one of Telmisartan or Duloxetine.

### Metformin for Stroke Protocol

Metformin has a formula C₄H₁₁N₅ and is well established as a main first-line medication for the treatment of type 2 diabetes, particularly in people who are overweight. It is also used in the treatment of polycystic ovary syndrome. Metformin is generally regarded as safe and well-tolerated.

Metformin is a biguanide drug that reduces blood glucose levels by decreasing glucose production in the liver, decreasing intestinal absorption, and increasing insulin sensitivity. Metformin decreases both basal and postprandial blood glucose levels. In PCOS, Metformin decreases insulin levels, which then decreases luteinizing hormone and androgen levels.

Metformin has an elimination half-life of 4-8.7 hours and the effective timing of Metformin as a medicament 20 (alone or in combination with the other medicaments 20) is within 4 hours of the implementing of a robotic assisted rehabilitation 30 session by the subject with the robot 40. More preferably Metformin is orally administered to the patient within 3 hours and most preferably within 2 hours (often about one hour) of the implementing of a robotic assisted rehabilitation 30 session by the subject with the robot 40.

The Metformin, like Cilostazol, may have several delivery methods or routes of administration, but oral is preferred. Effective amounts of Metformin in the protocol of the present invention (where the patient is not already taking this drug) is less than 850 Mg, more preferably less than 500 Mg, and most preferably less than 250 Mg per robotic assisted rehabilitation 30 session by the subject with the robot 40. Where the medicament 20 includes Metformin with one or more of the other cited drugs the effective amounts of Metformin in the protocol of the present invention (where the patient is not already taking this drug) is 125 Mg, possibly less than 125 Mg per robotic assisted rehabilitation 30 session by the subject with the robot 40.

As noted Metformin is often taken by Type 2 diabetics, and their dosage typically is initially 500-1000 Mg per day which is titrated up to 2000 Mg per day in a once per day time release oral treatment. Where a patient of the present protocol is taking 1000 Mg-2000 Mg of Metformin per day for other reasons, like type 2 diabetes, then this prescribed about is the effective amount for the protocol of the present invention and no additional Metformin is required, but this Metformin should still be taken within the effective time of the robotic assisted rehabilitation 30 session by the subject with the robot 40.

### Telmisartan for Stroke Protocol

Telmisartan has a chemical formula C₃₃H₃₀N₄O₂ is an angiotensin II receptor blocker that shows high affinity for the angiotensin II receptor type 1 (AT₁), with a binding affinity 3000 times greater for AT₁ than AT₂. In addition to blocking the renin-angiotensin system, Telmisartan acts as a selective modulator of peroxisome proliferator-activated receptor gamma (PPAR-γ), a central regulator of insulin and glucose metabolism. Telmisartan's dual mode of action may provide protective benefits against the vascular and renal damage caused by diabetes and cardiovascular disease (CVD). Telmisartan demonstrates activity at the peroxisome proliferator-activated receptor delta (PPAR-δ) receptor and activates PPAR-δ receptors in several tissues. Also, Telmisartan has a PPAR-γ agonist activity.

Telmisartan has an elimination half-life of 24 hours and the effective timing of Telmisartan as a medicament 20 (alone or in combination with the other medicaments 20) can be considered the same as Cilostazol for the purpose of the present invention. Telmisartan is preferably administered orally. Effective amounts of Telmisartan in the protocol of the present invention (where the patient is not already taking this drug) is less than 80 Mg, more preferably less than 40 Mg, and most preferably less than 20 Mg per robotic assisted rehabilitation 30 session by the subject with the robot 40. Where a patient of the present protocol is taking 20 Mg-80 Mg (or more) of Telmisartan per day for other reasons, like high blood pressure, then this prescribed about is the effective amount for the protocol of the present invention and no additional Telmisartan is required, but this Telmisartan should still be taken within the effective time of the robotic assisted rehabilitation 30 session by the subject with the robot 40.

### Duloxetine for Stroke Protocol

Duloxetine, having a chemical structure of C₁₈H₁₉NOS, is a serotonin-norepinephrine reuptake inhibitor. It is a medication used to treat major depressive disorder, generalized anxiety disorder, fibromyalgia, neuropathic pain and central sensitization.

Duloxetine has an elimination half-life of 12 hours and the effective timing of Duloxetine as a medicament 20 (alone or in combination with the other medicaments 20) can be considered the same as Cilostazol for the purpose of the present invention. Duloxetine is preferably administered orally. Effective amounts of Duloxetine in the protocol of the present invention (where the patient is not already taking this drug) is less than 60 Mg, more preferably less than 30 Mg, and most preferably less than 20 Mg per robotic assisted rehabilitation 30 session by the subject with the robot 40. Where a patient of the present protocol is taking 60 Mg (or more) of Duloxetine per day for other reasons then this prescribed about is the effective amount for the protocol of the present invention and no additional Duloxetine is required, but this Duloxetine should still be taken within the effective time of the robotic assisted rehabilitation 30 session by the subject with the robot 40.

### Robotic Assisted Rehabilitation 30 for Stroke Protocol

Robotic assisted rehabilitation 30, also called robot-mediated rehabilitation is an exercise-based therapy using robotic devices 40 that enable the implementation of highly repetitive, intensive, adaptive, and quantifiable physical training. Robotic systems 40 used in the field of neurorehabilitation under the protocol of the invention can be organized under two basic categories: exoskeleton and end-effector type robots. Exoskeleton robotic systems 40 allow accurate determination of the kinematic configuration of human joints, while end-effector type robots exert forces only in the most distal part of the affected limb. A growing number of commercial robotic devices 40 have been developed employing either configuration. Examples of exoskeleton type robots 40 include the ARMEO®SPRING, ARMEO®POWER, and MYOMO® brands and examples of end-effector type robots 40 include the INMOTION®, BURT®, KINARM™ and REAPLAN®. Both categories of robots 40 enable the implementation of intensive training and objective review of results allowing for effective implantation of the protocol of the present invention.

With respect to the operation of prosthetic limbs, it has been shown that the human body generates synaptic pathways in response to a newly stimulated operation, e.g., operating a prosthetic arm. Furthermore, this form of rehabilitation enhances the precise timing and coordination of multiple inter-related sub-processes. The proposed method in this application enhances and accelerates the recovery of function and may minimize the necessary degree of implanted artificial stimuli with deliberate increases, or additional stimuli, to underlying neuroplasticity, synaptogenesis, and or neurogenesis (Neuroplasticity).

The individual therapy or patient motion for the protocol of the invention is determined by the robot 40 and is generally known in the art. For upper limb therapy the INMOTION® robotic therapy on INMOTION® robot 40 is believed to enhance the therapist ability to drive repetition and neuroplasticity within the protocol of the present invention. The protocol of the present invention preferably includes, for upper limb therapy, using robotic devices 40 over 30-60-min individual sessions each having 500-700 repetitions of defined motion for a total session time of 16 hours spaced over time with 3-5 sessions per week. This amount is safe despite the larger number of movement repetitions in each session.

In one example of the present invention implements Robotic assisted rehabilitation 30 in the form of a prosthetic glove (soft, under-actuated and compliant robotic exo-gloves) assists patient moving hand that sends signal to brain implants. Specifically, the subject is given the exo-glove that both moves his hand independently and augments any small muscle movement he is able to initiate on his own. This repetitive stimulation is transmitted to the left motor cortex area of the brain corresponding to the right hand. With enough stimulation this movement alone induces neurogenesis and helps to overcome the user fatigue inherent in the 500-700 reps implemented in a session. In the presence of pharmacological agents or medicaments 20 designed to enhance neurogenesis this therapy is very effective.

This technique of the present invention could be augmented by either external or internal Near Infrared phototherapy, Electromagnetic stimulation or direct Transcranial electrical stimulation, which are detailed below as independent therapies.

This protocol is feasible and believed that medicament 20 enhanced robotic therapy of the protocol will lead to significantly more improvement in impairment as compared to conventional usual care. The protocol's enhanced robotic training is believed to be more effective in reducing motor impairment than conventional robotic training alone. The protocol is believed to avoid any significant increase of muscle hyperactivity and shoulder pain due to the intensive training. The intensive robotic training with the protocol may improve activities of daily living after stroke

Intensity is an ingredient in an effective post-stroke motor rehabilitation program in the protocol of the present invention. Significant changes in motor performance are believed to result from intensive training and the protocol should should contain at least 10 h-16 h of exercise-based interventions or sessions to induce significant effects on activities of daily living. The pharmaceutical based protocol of the invention is believed to maintain patients' active participation throughout the protocol and sessions thereof even as the robot reduces the assistance, thereby demanding more active patient participation. Moreover, as the robotic assistance in the protocol of the invention is decreased from session to session, yielding a further increase in active participation over sessions.

A representative Robotic assisted rehabilitation 30 plan for the protocol of the present invention is sixteen robot 40 assisted, 45-minute sessions scheduled 4-days per week performing an average of 650 movements per session.

The robotic devices 40 enable an easy quantification of the dose administered within a training session. Robotic devices 40 offer patients various forms of feedback (visual, auditory, haptic . . . ) and provide patients with different forms of knowledge of results (how many successes) or of their motor performance (number of repetitions, amount of assistance, and deviation from straight lines . . . ). This feedback information can not only optimize patient's motivation and engagement but can also enhance learning and recovery. Rehabilitation robots 40 used in the protocol are remarkably good evaluation tools, allowing an accurate characterization and quantification of time-course evolution of motor performance. Most advanced robotic systems or devices 40 include sensors which measure and record kinematic and kinetics during upper extremity movement used to derive indicators and movement features. Kinematic indicators may be used as valid objective measures for assessing upper limb motor impairments and this data could complement clinical assessment. Kinematic measurements, such as the active range of motion (AROM), might be a reliable indicator of motor recovery.

The protocol may also consist of a series of robot-training sessions interspaced by sessions in which the clinicians assist patients to translate their impairment gains into function. In this implementation the robotic therapy focuses on impairment with the therapist then tailoring therapy to the particular patient's need and assisting in translating impairment gains into function.

### Alternative Disorders and Conditions and Desired Effects

The protocol of the present invention is not limited to the stroke protocol outlined above and following is a list of disorders and conditions which may benefit from the more broadly proposed method, and for which rehabilitation is appropriate and feasible. These include Prosthetic limb operation; non stroke Limb rehabilitation/enhancement; Traumatic Brain injury, mild, moderate or severe and concussion; Brain injury due to exposure to neurotoxins; Learning enhancement such as language or mathematics, hemorrhage Stroke; Alzheimer's Disease; Parkinson's Disease; Frontotemporal Dementias; Huntington's Disease; Amyotrophic lateral sclerosis (ALS); Multiple Sclerosis (MS) and Cerebral palsy (CP)

### Alternative Therapeutic Intervention List

The medicaments 20 can expand from the above listing that is preferred for stroke when applying the method to other conditions. For example, one manifestation of the method will be the use of electric/electromagnetic stimuli in combination with fluoxetine, sertraline, or other SSRI-categorized medication, in the context of prosthetic limb operations. Studies have demonstrated that antidepressants, including SSRIs, induce a form of plasticity that is similar in important respects to plasticity seen in juvenile neuronal networks. Particularly these medications have been shown to increase plasticity in hippocampal dentate gyrus (DG) cells, which is a well-documented site underlying new learning. Further research has demonstrated increases in neuroplasticity or markers of neuroplasticity in other areas such as the visual cortex, amygdala, and medial pre-frontal cortex. In patients with mood disorders, SSRIs have been shown to be most effective when combined with other therapy, e.g., Cognitive-Behavorial, CBT, supporting the potential benefit of combining neuroplasticity (from SSRIs) with a targeting intervention (CBT). The method described in this patent employs the same physiological principles, but using an intervention that exercises more fundamental neuro-behavioral systems. The following is a list of SSRIs suitable for the methodology of the present invention: Citalopram; Escitalopram; Fluoxetine; Fluvoxamine; Paroxetine; Sertraline; and Vilazodone.

Another manifestation of the method will be to use electrical stimuli from an implanted device or external electromagnetic stimulator in combination with pharmacological therapies designed to enhance Brain Derived Neuro-trophic factor (BDNF) action in promoting neuroplasticity. BDNF signaling (particularly through its TrkB receptor target) forms a critical component in multiple types of neuroplasticity-enhancing interventions. Evidence suggests that its expression is influenced by increased neural activity, which rehabilitation tasks are meant to provide. BDNF enhancing therapeutics include, but are not limited to: ketamine and its metabolic derivatives norketamine and hydroxynorketamine (HNK); memantine; riluzole; Quercetin; Therapeutic administration of botanicals with BDNF effect, e.g., ginsenosides, salidroside, glycosides, Ginkgo biloba, Hypericum perforatum; Artesunate; and Clemastine

Another manifestation of the method will be the use of electrical/electromagnetic stimuli or evoked potentials in combination with steroids, such as: Neurosteroids (Pregnenolone, Dehydroepiandrosterone, Allopregnanolone, and their synthetic analogs). Neurosteroids can affect neuroplasticity and neurogenesis through their actions on DNA gene transcription and possibly more directly through neurotransmitter receptors and receptor modulation; Sex steroids, i.e. testosterone, estrogen, and progesterone. These steroids have strong effects on general neuroplasticity, and this manifestation of the method incorporates their potential benefit in rehabilitative therapy.

Another manifestation of the method will be the use of electrical stimuli or evoked potentials in combination with pharmacological psychedelics, which have been shown to promote neuroplasticity both structurally and functionally, including but not limited to: tryptamines (N,N-dimethyltryptamine [DMT] and psilocin); amphetamines (2,5-dimethoxy-4-iodoamphetamine [DOI] and MDMA); and ergolines (lysergic acid diethylamide [LSD]).

Multiple methods of inducing Neuroplasticity are discussed below. The neuroplasticity medicament includes those described in WO 2020/097320 which medicaments are incorporated herein by reference and may be summarized as comprising at least one of an anti-depressant, a Brain Derived Neurotrophic Factor enhancer, a steroid, a psychedelic, valproic acid, NDRI's, lithium carbonate, Metformin, N-Acetylcystine, and Human Growth Hormone.

Another manifestation of the method will be the use of electrical stimuli or evoked potentials in combination with other therapeutic agents and methods not mentioned above, that induce neuroplasticity and neurogenesis, including: Stem cells, Exosomes and other cellular therapies; Valproic Acid; Non-SSRI antidepressants; NDRI's, lithium carbonate, heterocyclic antidepressants, N-Acetylcystine, Human Growth Hormone; Selective Norepinephrine and Serotonin Reuptake Inhibitors (SNRIs) including Desvenlafaxine, Levomilnacipran, Milnacipran and Venlafaxine; Tricyclic and Heterocyclic Antidepressants including Amitriptyline, Amoxapine, Desipramine, Doxepin, Imipramine, Nortriptyline, Protriptyline, Trimipramine, Trazodone and, Maprotiline; Dopamine and mixed Dopamine and Serotonin Reuptake Inhibitors including Bupropion, Amineptine, Ethylphenidate, Modafinil, Armodafinil, Vanoxerine, Amantadine, Benztropine, Methylphenidate, and Rimcazole; Tetracyclic Antidepressants such as Mirtazipine and Maprotiline; Monoamine Oxidase Inhibitors including Isocarboxazid, Phenelzine, Rasagiline, Selegiline, Tranylcypromine and Methylene Blue; Anticonvulsants such as Valproic Acid and Ethosuximide; Plant and Fungi Derived Substances including Curcumin, Aristoforin, Quercetin, Artesunate, N-Acetylcysteine, Lion's Mane Mushroom extract, Psilocin, Mescaline, LSD, DOI, and DMT; Muscarinics including Galantamine, Rivastigmine, Memantine, and Donepezil; Cannabidiol; NMDA Receptor Antagonist family including NMDA, Ketamine, Esketamine, Norketamine, HNK, NV-5138 Leucine, Apimostinel and Rapastinel; Immunomodulators such as NeuroStat, Cyclosporine A, Dexamethasone, Naltrexone, Naloxone, Prednisolone, Methylprednisolone, and Fluticasone; Hormone Based Medications such as Progesterone, Estrogens, Flavinoids, Resveratrol, Human Growth Hormone (various isomers), Ghrelin and Melatonin; Anti-Inflammatory Drugs such as Antithrombin, Celecoxib, Dexamethasone; Lithium; Minocycline; Anti Nogo antibodies; Bacterial Enzyme Chondroitinase ABC (ChABC); Bacterial Toxin VX-210; Metalloproteinases and MMP9 antibodies and inhibitors

### Non-Robotic Device Based Tasks for Tsr-Nre

The protocol of the present invention when expanded beyond stroke as discussed above can be implemented with alternative stimuli or tasks. These new stimuli of the present invention, include the growing use of electrical and electromagnetic stimuli within the nervous system to address neuro-dysfunction resulting from limb loss, neurological trauma or neurodegenerative diseases. One goal of the method of the inventionis to enhance acquisition or re-acquisition of lost or medically valuable neurological function through adaptation to therapeutic tasks, i.e., during the performance or learning of skills for medical purposes. This enhancement can occur at the interface of medical devices within body tissues and at the internal site of action of externally applied electrical and electromagnetic devices. In the various tasks, the participant engages in various cognitive, sensory, neuro-motor other therapeutic neurological activities. In the presence of a neuroplasticity enhancing therapy the participants are instructed to execute a task aligned with the functions associated with the electrical or electromagnetic implant or device. The monitoring equipment tracks and measures the effects of this task both from the perspective of neurological recovery and the effectiveness of the electrical device or electromagnetic stimuli.

The means of evaluating the effect of this combinatorial therapy include but are not restricted to objective biomarkers such as neuroimaging (MRI, fMRI, PET, intracranial blood flow), serum biomarkers, oculomotor testing, neuropsychiatric and cognitive testing, neuromuscular and sensory performance etc.

The following represent alternative examples of the present protocol beyond the stroke implementation discussed above.

### Rtms Based Addiction Treatment, Depression Treatment and Mtbi Treatment Methodology Examples

Repetitive transcranial magnetic stimulation (rTMS) is a form of brain stimulation therapy currently used to treat depression and anxiety. The therapy involves using a magnet to target and stimulate certain areas of the brain. Present invention implements this process with subjects with pharmacologically induced neuroplasticity process for treatment of drug addiction, such as in particular nicotine addiction. The relevant neuroplasticity inducing drugs are outlined above.

Success of the treatment may be quantified by obtaining and evaluating at least one of the following metrics: days between cravings, days till cessation, decrease in desire for cigarettes, number of cigarettes consumed per day or week.

In another example of the present invention the present invention implements a repetitive transcranial magnetic stimulation (rTMS) process with subjects with pharmacologically induced neuroplasticity for treatment of depression, such as in subjects exhibiting suicidal tendencies. The relevant neuroplasticity inducing drugs are outlined above. Success of the treatment may be quantified by obtaining and evaluating the following metrics: suicidality and functionality (how able are you to get through your day).

In another example of the present invention, the present invention implements a repetitive transcranial magnetic stimulation (rTMS) process with subjects with pharmacologically induced neuroplasticity for treatment of mTBI. The relevant neuroplasticity inducing drugs are outlined above. Success of the treatment may be quantified by obtaining and evaluating PTSD biomarkers and/or the NKI Concussion Score™ (offered by Neurolign). Alternatively balance plate feedback, dual track gait analysis, and pupil responses could be used to access progress of treatments.

### Neuronal Oscillations Producing Brainwaves Graft

In one example of the present invention, the present invention replicates neural correlates neuronal oscillations producing brainwaves for proper limb motion and grafts these onto the patient subjects with pharmacologically induced neuroplasticity. Dr. Poltorak of Neuroenhancement Labs, LLC has outlined this basic operation of grafting neuronal oscillations producing brainwaves in subjects. In the present invention this operates in rehabilitation of stroke victim limbs by having the subject, for example, manipulate their “good” hand and an EEG would record activity within the good hand and create a mirror image for acting on movement of a damaged hand in the patient with pharmacologically induced neuroplasticity. Alternatively, an EEG can record activity within the good hand of a separate donor and create a brain wave pattern for acting on movement of a damaged hand in the patient with pharmacologically induced neuroplasticity. Essentially the invention will record an EEG of a healthy response and graft it (or mirror image of it) into the relevant brain portion of a patient with pharmacologically induced neuroplasticity during treatment. The therapy includes repeating the grafted movement in the rehabilitating limb for up to 600-700 times as discussed above with the robot assisted therapy. This therapy could also be integrated into the robot assited therapy detailed above.

### Near Infrared Phototherapy Examples

In one example of the present invention the invention implements near infrared light therapy in patients with pharmacologically induced neuroplasticity. Consider, in particular in a stroke victim in which an area of the brain suffers injury from transient hypoxia for a variety of reasons. The injured area has cells that recover promptly, cells that may never recover, and a watershed area where cells are senescent and may recover with the correct treatment/stimuli. There is also at least some potential for neurogenesis to create new cells in the injured area. Near Infrared light can transmit energy to these cells that they can use to make it easier for them to create ATP, the energy currency of the cell. This energy allows some cells to regain function and facilitates neurogenesis. The light energy sourced can now be implanted into damaged areas of the brain to deliver the energy more efficiently. It is noted that this type of intracranial light stimulation has also been used in animal studies to control the movement of body parts like limbs and whiskers, and has been used to treat aphasia in human stroke victims. As discussed above, neurogenesis in a targeted part of the brain in the methods of the present invention can be induced with repetitive neuromuscular exercises in subjects with pharmacologically induced neuroplasticity in less than 600 repetitions. The use of implanted Near Infrared devices offers the possibility of further facilitating this both by adding energy to the target area and by repetitive stimulation. The simultaneous application of neurogenesis enhancing pharmacology act synergistically with an implanted (or externally acting) near infrared phototherapy device. Further, simultaneous stimulation with a physiological treatment known to enhance the release of BDNF such as acute intermittent hypoxia can be expected to add even more therapeutic potential. The methodology improves the ability to awaken senescent cells, encourage the growth of new cells in the area, and form new neural connections to restore function lost from the stroke. This methodology could also be implemented with the above described robot assisted therapy as noted earlier.

### Acupuncture Electrotherapy Based Examples

Acupuncture needles can be used as electrical stimulators via frequency (e.g. electrical on-off patterns) in what is known as acupuncture electrotherapy. The company Scrip Hessco offers a line of commercially available acupuncture electrotherapy machines. The acupuncture methodologies integrate 5000 years of knowledge of location of peripheral nerves. Broadly the present invention contemplates performing specific acupuncture electrotherapy methodologies in patients with pharmacologically induced neuroplasticity. The simultaneous application of neurogenesis enhancing pharmacology act synergistically with acupuncture electrotherapy. A further variation of this example of the present invention would be to track the destination points in the brain that acupuncture signals go to and then implant devices to receive those impulses and respond as desired. Alternatively, the method may implant a long-term source of medication or cells right at the destination spot via a slow release gel, bead, nanoparticle or other small vehicle. For example, the method may place a sensor/device in the brain that hold a neuro-stimulatory substance (medication, lights, electrodes, stem cells) and use peripheral stimulation to verify it in the right place. When the placement is confirmed the method will signal it to release the substance in a desired manner (e.g., all at once, pulses, time delayed etc.).

### Dbs Examples

As noted above, Deep brain stimulation (DBS) is a neurosurgical procedure involving the placement of a medical device called a neuro-stimulator (sometimes referred to as a “brain pacemaker”), which sends electrical impulses, through implanted electrodes, to specific targets in the brain (brain nuclei). In one example of the present invention the invention implements DBS for the treatment of movement disorders, including Parkinson's disease, essential tremor, and dystonia in patients with pharmacologically induced neuroplasticity. One aspect of this methodology measures Dopamine release as a biomarker of treatment efficacy. The simultaneous application of neurogenesis enhancing pharmacology act synergistically with the neuro-stimulator.

### Ces Example

As noted above, Cranial electrotherapy stimulation (CES) is a form of neurostimulation that delivers a small, pulsed, alternating current via electrodes on the head. In one aspect of the invention the present invention implements CES used on patients with pharmacologically induced neuroplasticity for treating conditions such as anxiety, depression and insomnia. CES based method according to the invention have potential as a possible treatment for headaches, fibromyalgia, smoking cessation, and opiate withdrawal. The presence of pharmacological agents operate synergistically with the CES treatments.

### Vns Example

As discussed above, Vagus nerve stimulation (VNS) is a medical treatment that involves delivering electrical impulses to the vagus nerve. The present invention contemplates one embodiment using VNS as treatment for intractable epilepsy in patients with pharmacologically induced neuroplasticity.

### Rns Example

Responsive neurostimulation (RNS) is a surgical approach to treating seizures wherein a neurostimulator is placed under the scalp and within the skull, and it is connected to 2 electrodes placed either on the surface of the brain, into the brain, or a combination of both. The device continuously monitors brain activity and then is programmed to detect seizures. When a seizure or seizure-like activity is detected, the device delivers a small amount of electrical current to the brain to stop or shorten the seizure, or possibly prevent a seizure altogether, the present method adds the release of a pharmacological agent which induces neuroplasticity with the RNS treatment.

### Ms and Als Treatment Examples

Both MS and ALS could be treated with stimulation of the skin (peripheral body locations) and brain in one method of the present invention in patients with pharmacologically induced neuroplasticity. Existing ALS or MS biomarkers can be tracked for evaluation and modification of the therapy. Neuromuscular stimulation of peripheral nerves, such as stimulation with heat (or cold), electrical frequency, is applied to stimulate growth of myelin sheath in patients taking neuroplasticity drugs. The presence of pharmacological agents operate synergistically with the skin and brain stimulation.

### Extracorporeal Shock Wave Therapy Example

Extracorporeal shock wave (ECSW) was originally developed for the treatment of lithotripsy. Some data suggests ECSW therapy is effective for improving acute interstitial cystitis, chronic tendinitis, delayed fracture healing with promising results, attenuate DM-induced diabetic neuropathy, and attenuating neuropathic pain. The present invention, in one embodiment, provides a methodology for treating stroke with the combination of ECSW therapy and effective amounts of N-Acetylcysteine (NAC). NAC has been shown to attenuate severe blast injuries and these amounts and delivery mechanisms are believed to be appropriate for stroke treatment methodologies of the present invention.

### CONCLUSION

While this invention has been particularly shown and described with references to the preferred embodiments thereof, specifically the preferred embodiment of the present invention is directed to ischemic stroke rehabilitation protocol implementing robotic assisted rehabilitation in subjects with pharmacologically induced neuroplasticity, it will be understood by those skilled in the art that various changes in form and details may be made therein without departing from the scope of the invention. The scope of the present invention is set forth in the following claims and equivalents thereto.

What is claimed is:

1. A stroke rehabilitation protocol comprising the steps of:
a) providing a subject with medicaments including at least one of Cilostazol, Metformin, Telmisartan and Duloxetine;
b) implementing robotic assisted rehabilitation in the subject with pharmacologically induced neuroplasticity, wherein the medicaments are supplied in effective amounts and timing whereby the medicaments induce neuroplasticity during the robotic assisted rehabilitation.

2. The stroke rehabilitation protocol according to claim 1, wherein the medicament includes Cilostazol.

3. The stroke rehabilitation protocol according to claim 2, wherein implementing robotic assisted rehabilitation includes multiple robotic assisted rehabilitation sessions and Cilostazol is orally administered to the patient within 4 hours implementing of each robotic assisted rehabilitation session.

4. The stroke rehabilitation protocol according to claim 2, wherein implementing robotic assisted rehabilitation includes multiple robotic assisted rehabilitation sessions and Cilostazol is orally administered to the patient within 2 hours implementing of each robotic assisted rehabilitation session.

5. The stroke rehabilitation protocol according to claim 2, wherein implementing robotic assisted rehabilitation includes multiple robotic assisted rehabilitation sessions and Cilostazol is orally administered to the patient about one hour before implementing of each robotic assisted rehabilitation session.

6. The stroke rehabilitation protocol according to claim 2, wherein implementing robotic assisted rehabilitation includes multiple robotic assisted rehabilitation sessions and Cilostazol is orally administered to the patient in amounts of less than 50 Mg per robotic assisted rehabilitation session.

7. The stroke rehabilitation protocol according to claim 2, wherein implementing robotic assisted rehabilitation includes multiple robotic assisted rehabilitation sessions and Cilostazol is orally administered to the patient in amounts of about 25 Mg per robotic assisted rehabilitation session.

8. The stroke rehabilitation protocol according to claim 2, wherein the medicament includes Metformin.

9. The stroke rehabilitation protocol according to claim 9, wherein implementing robotic assisted rehabilitation includes multiple robotic assisted rehabilitation sessions and Cilostazol and Metformin are orally administered to the patient within 4 hours implementing of each robotic assisted rehabilitation session.

10. The stroke rehabilitation protocol according to claim 9, wherein implementing robotic assisted rehabilitation includes multiple robotic assisted rehabilitation sessions and Cilostazol and Metformin are orally administered to the patient within 2 hours implementing of each robotic assisted rehabilitation session.

11. The stroke rehabilitation protocol according to claim 9, wherein implementing robotic assisted rehabilitation includes multiple robotic assisted rehabilitation sessions and Metformin is orally administered to the patient in amounts of less than 250 Mg per robotic assisted rehabilitation session.

12. The stroke rehabilitation protocol according to claim 2, wherein the medicament includes one of Telmisartan and Duloxetine.

13. The stroke rehabilitation protocol according to claim 12, wherein implementing robotic assisted rehabilitation includes multiple robotic assisted rehabilitation sessions and Cilostazol and one of Telmisartan and Duloxetine are orally administered to the patient within 4 hours implementing of each robotic assisted rehabilitation session.

14. The stroke rehabilitation protocol according to claim 12, wherein implementing robotic assisted rehabilitation includes multiple robotic assisted rehabilitation sessions and Cilostazol and one of Telmisartan and Duloxetine are orally administered to the patient within 2 hours implementing of each robotic assisted rehabilitation session.

15. The stroke rehabilitation protocol according to claim 9, wherein implementing robotic assisted rehabilitation includes multiple robotic assisted rehabilitation sessions and one of Telmisartan and Duloxetine is orally administered to the patient in amounts of less than 20 Mg per robotic assisted rehabilitation session.

16. The stroke rehabilitation protocol according to claim 1, wherein the medicament includes one of Telmisartan and Duloxetine.

17. The stroke rehabilitation protocol according to claim 1, wherein the medicament includes Metformin.

18. The stroke rehabilitation protocol according to claim 1, wherein implementing robotic assisted rehabilitation includes multiple robotic assisted rehabilitation sessions on an upper limb robot with 500-700 repetitions per session and wherein implementing robotic assisted rehabilitation includes the multiple robotic assisted rehabilitation sessions which total at least 10 hours.

19. A stroke rehabilitation protocol comprising the steps of:
a) providing a subject with oral medicaments including at least one having a chemical formula of i) C₂₀H₂₇N₅O₂, ii) C₄H₁₁N₅, iii) C₃₃H₃₀N₄O₂ and iv) C₁₈H₁₉NOS;
b) implementing robotic assisted rehabilitation in the subject with pharmacologically induced neuroplasticity, wherein the medicaments are supplied in effective amounts and timing whereby the medicaments induce neuroplasticity during the robotic assisted rehabilitation.

20. A stroke rehabilitation protocol comprising the steps of:
a) providing a subject with oral medicaments including at least one having a chemical formula of i) C₂₀H₂₇N₅O₂, ii) C₄H₁₁N₃, iii) C₃₃H₃₀N₄O₂ and iv) C₁₈H₁₉NOS;
b) implementing multiple robotic assisted rehabilitation sessions in the subject with pharmacologically induced neuroplasticity, wherein the medicaments are supplied in effective amounts and timing whereby the medicaments induce neuroplasticity during each of the robotic assisted rehabilitation session, and wherein the medicaments are orally administered to the patient within 2 hours implementing of each robotic assisted rehabilitation session.
